(19) United States
(12) Patent Application Publication
(10) Pub. No.: US 2026/0345014 A1
(43) Pub. Date: Nov. 5, 2026

(54) Systems and Methods for Determining Cardiovascular Parameters from Pulmonic Measurements

(71) Applicant: Becton, Dickinson and Company, Franklin Lakes, NJ (US)

(72) Inventors: Monera Amani Kamal, Orange, CA (US); Zhongping Jian, Irvine, CA (US); Jacobus Jozef Gerardus Maria Settels, De Hoef (NL); Feras Al Hatib, Irvine, CA (US)

(21) Appl. No.: 19/487,145

(22) PCT Filed: May 28, 2024
(86) PCT No.: PCT/US2024/031282

§ 371 Date: Nov. 24, 2025

Related U.S. Application Data
(60) Provisional application No. 63/505,677, filed on Jun. 1, 2023.

(51) Int. Cl. A61B 5/02; A61B 5/00; A61B 5/021; A61B 5/0215; A61B 5/026; A61B 8/04; A61B 8/06

(52) U.S. Cl. CPC A61B 5/02028; A61B 5/02108; A61B 5/0215; A61B 5/026; A61B 5/4839; A61B 8/04; A61B 8/065; A61B 2562/0247

(57) ABSTRACT

Systems and methods arc for evaluating preload responsiveness utilizing hemodynamic data that is derived from a continuous blood pressure or blood flow sensor representative of right-side measurements from the heart. Hemodynamic data features can be derived from a blood pressure or blood flow waveform and utilized to determine hemodynamic parameters to guide fluid resuscitation treatment and/or maintain the health and wellbeing of a patient that, such as a patient under monitoring during a surgery. In one example, beat-to-beat right-side stroke volume variation (SVV) is determined as a function of the standard deviation of the blood pressure waveform over a plurality of cardiac cycles.

### CROSS REFERENCE TO RELATED APPLICATIONS

This application claims priority to U.S. Provisional Patent Application No. 63/505,677, entitled Systems and Methods for Determining Cardiovascular Parameters from Pulmonic Measurements, filed Jun. 1, 2023, the disclosure of which is herein incorporated by reference.

### TECHNOLOGICAL FIELD

The disclosure is generally directed to systems and methods for determining blood parameters from right-side measurements of the heart, and more specifically for determining stroke volume variation, pulse pressure variation, and/or systolic pressure variation utilizing sensors within the right-side measurements from the heart.

### BACKGROUND

Patient hemodynamic monitoring typically comprises monitoring of blood pressure and other parameters, such as Cardiac Output (CO), Stroke Volume (SV), and Stroke Volume Variation (SVV), which can be derived from blood pressure waveforms. Accurately calculating those parameters is of great importance, as it can assist clinicians in assessing patient health and informing treatment options.

Fluid resuscitation (also referred to fluid replacement or fluid administration) is the delivery of fluids (e.g., intravenous delivery of saline) to a patient, which can help if a patient is hypovolemic (i.e., low on circulating fluids). Hypovolemia results in poor tissue perfusion and may cause tissue damage and vital organ dysfunction. Correct clinical assessment of hypovolemia in hemodynamically unstable patients can be difficult. Specifically, it is very difficult to predict whether a hemodynamically unstable patient will respond to fluid therapy. Moreover, hypervolemia (i.e., excessive circulating fluids) can cause significant pulmonary or cardiac dysfunction. In consideration of these factors and risks, the decision to perform fluid resuscitation as a treatment is critical. Fluid responsiveness (also referred to as volume responsiveness and preload responsiveness) is the ability to increase stroke volume in response to fluid administration, and is a major and important determinant to assess the appropriateness of fluid resuscitation therapy in order to improve cardiac performance and organ perfusion.

Many hemodynamic parameters based on ventricular preload have been used as predictors of fluid responsiveness. For example, right arterial pressure (RAP) and pulmonary artery occlusion pressure (PAOP) are commonly used in the intensive care unit (ICU) when deciding whether fluid resuscitation is appropriate. Other hemodynamic parameters of ventricular preload that can be utilized include right ventricular end diastolic volume (RVEDV) and left ventricular end diastolic area (LVEDA), which can be measured with transesophageal echocardiography. Several studies and case reports have concluded, however, that these static parameters have poor predictive value of fluid responsiveness.

Several studies have confirmed the clinical significance of monitoring variations of left ventricular stroke volume under mechanical ventilation. Stroke volume variation (SVV) is caused by cyclic increases and decreases of intrathoracic pressure due to mechanical ventilation, which results in variation of the cardiac preload and afterload. SVV has recently been extensively investigated and several studies have shown the usefulness of using SVV as a predictor of fluid responsiveness in various clinical situations. Several other parameters related to SVV have also been found to be useful as well. In particular, systolic pressure variation (SPV), along with its delta-Up (ΔUp) and delta-Down (ΔDown) components, have been found to be a useful predictor of fluid responsiveness. SPV is based on the changes in the arterial pulse pressure due to respiration-induced variations in stroke volume. Pulse pressure variation (PPV) has also been demonstrated to be a valid predictor of fluid responsiveness.

The specific monitoring of SVV has both specific difficulties and advantages. Physiologically, SVV is based on several complex mechanisms of cardio-respiratory interaction. Changes in left ventricular preload can lead to distinct variations in left ventricular stroke volume and systolic arterial pressure. Monitoring of SVV enables prediction of left ventricular response to volume administration and helps with correct assessment of hypovolemia and the subsequent decision to perform volume resuscitation in many critical situations.

### SUMMARY

Systems and methods for hemodynamic monitoring can comprise utilization of a right-side pressure sensor to generate an arterial waveform. Hemodynamic data features can be extracted from the waveform. The extracted hemodynamic data features can be utilized in a computational model to identify specific parameters that can guide treatment.

In some aspects, the techniques described herein relate to a method for computing a preload responsiveness indicator, including obtaining a waveform from a subject's right heart circulation, wherein the waveform includes a plurality of heart beats, and wherein the waveform represents at least one of blood pressure and blood flow, identifying individual heart beats in the pressure or flow waveform, obtaining a hemodynamic variable for each identified heart beat in the waveform, and computing a preload responsiveness indicator using the obtained hemodynamic variable.

In some aspects, the techniques described herein relate to a method, wherein the waveform is obtained from at least one of a right ventricle, a right atrium, a pulmonary artery, and a pulmonary vein.

In some aspects, the techniques described herein relate to a method, wherein the waveform is obtained using a pulmonary artery catheter or a right ventricular catheter.

In some aspects, the techniques described herein relate to a method, wherein the waveform is a blood flow waveform measured from a right ventricle or a pulmonary artery.

In some aspects, the techniques described herein relate to a method, wherein the blood flow waveform is measured using an invasive ultrasound probe.

In some aspects, the techniques described herein relate to a method, wherein the waveform is a blood flow waveform computed from a right ventricular blood pressure waveform or a pulmonary artery blood pressure waveform.

In some aspects, the techniques described herein relate to a method, wherein the blood flow waveform is computed using a model of an input impedance of pulmonary circulation.

In some aspects, the techniques described herein relate to a method, wherein the model of the input impedance of the pulmonary circulation is based on a physiological Windkessel model.

In some aspects, the techniques described herein relate to a method, wherein the model of the input impedance of the pulmonary circulation is a machine learning model.

In some aspects, the techniques described herein relate to a method, wherein the waveform is obtained in real-time.

In some aspects, the techniques described herein relate to a method, wherein the waveform represents a time period of at least 5 seconds.

In some aspects, the techniques described herein relate to a method, wherein obtaining the waveform occurs on a continuous basis.

In some aspects, the techniques described herein relate to a method, wherein the obtained hemodynamic variable is selected from right ventricular stroke volume, right ventricular pulse pressure, right ventricular systolic pressure, pulmonary artery pulse pressure, pulmonary artery systolic pressure.

In some aspects, the techniques described herein relate to a method, wherein the obtained hemodynamic variable is right ventricular stroke volume, wherein right ventricular stroke volume is defined as proportional to a standard deviation of pulmonary arterial pressure of that beat.

In some aspects, the techniques described herein relate to a method, wherein the obtained hemodynamic variable is right ventricular stroke volume, wherein right ventricular stroke volume for each beat is defined as proportional to a standard deviation of pulmonary arterial pressure of that beat normalized by a mean pulmonary arterial pressure of that beat.

In some aspects, the techniques described herein relate to a method, wherein obtaining a hemodynamic variable includes filtering the hemodynamic variables.

In some aspects, the techniques described herein relate to a method, wherein filtering includes one or more of removing beats whose values of the hemodynamic variable are at least two standard deviations greater than or at least two standard deviations below its mean, removing beats whose values of the hemodynamic variable are in a highest 5% of data, and removing beats whose values of the hemodynamic variables are in a lowest 5% of data.

In some aspects, the techniques described herein relate to a method, wherein computing a preload responsiveness includes identifying a maximum value (max) of the hemodynamic variable, identifying a minimum value (min) of the hemodynamic variable, calculating a mean value (mean) of the max and the min, and computing the preload responsiveness indicator as

$$\frac{max - min}{mean} \times 100 .$$

In some aspects, the techniques described herein relate to a method, wherein the max is a largest value of the hemodynamic variable.

In some aspects, the techniques described herein relate to a method, wherein the max is an average of at least two largest values of the hemodynamic variable.

In some aspects, the techniques described herein relate to a method, wherein the min is a smallest value of the hemodynamic variable.

In some aspects, the techniques described herein relate to a method, wherein the min is an average of at least two smallest values of the hemodynamic variable.

In some aspects, the techniques described herein relate to a method, wherein computing a preload responsiveness includes identifying a response pattern in the hemodynamic variable by bandpass filtering the hemodynamic variable, identifying a maximum value (max) from the identified response pattern, identifying a minimum value (min) from the identified response pattern, calculating a mean value (mean) of the max and the min, and computing the preload responsiveness indicator as

$$\frac{max - min}{mean} \times 100.$$

In some aspects, the techniques described herein relate to a method, wherein the max is a largest value of the response pattern.

In some aspects, the techniques described herein relate to a method, wherein the max is an average of at least two largest values of the response pattern.

In some aspects, the techniques described herein relate to a method, wherein the min is a smallest value of the response pattern.

In some aspects, the techniques described herein relate to a method, wherein the min is an average of at least two smallest values of the response pattern.

In some aspects, the techniques described herein relate to a method, wherein computing a preload responsiveness includes identifying a response pattern in the hemodynamic variable by bandpass filtering the hemodynamic variable, wherein the bandpass filtering possesses a frequency range, identifying a modulation pattern in the waveform, wherein the modulation pattern is associated with a stimulus that affects blood pressure or blood flow, computing a power density spectrum of the hemodynamic variable from the identified heart beats in the modulation pattern, and computing the preload responsiveness indicator as a mean value (mean) of an amplitude of the power density spectrum over the frequency range.

In some aspects, the techniques described herein relate to a method, wherein the stimulus is selected from one or more of fluid infusion, vasopressor infusion, vasodilator infusion, spontaneous respiration, and mechanical respiration.

In some aspects, the techniques described herein relate to a method, wherein the frequency range is approximately 0.08 Hz to approximately 0.33 Hz.

In some aspects, the techniques described herein relate to a method, wherein the plurality of heart beats represents at least one respiratory cycle.

In some aspects, the techniques described herein relate to a method, further including providing a medical intervention to a subject or providing instructions to provide a medical intervention to the subject.

In some aspects, the techniques described herein relate to a method, wherein the medical intervention includes one or more of providing a fluid bolus, providing mechanical ventilation, and a pharmacological agent.

In some aspects, the techniques described herein relate to a method, wherein the pharmacological agent is selected from a vasopressor and a vasodilator.

In some aspects, the techniques described herein relate to a system for determining a preload responsiveness indicator, including a pressure or flow sensing device configured to sense a right heart blood pressure or flow of a subject, and a controller in communication with the pressure or flow sensing device, the controller including at least one processor and a memory device configured to store instructions, which instructions when executed instruct the controller to obtain a waveform from a subject's right heart circulation, wherein the waveform includes a plurality of heart beats, and wherein the waveform represents at least one of blood pressure and blood flow, identify individual heart beats in the pressure or flow waveform, obtain a hemodynamic variable for each identified heart beat in the waveform, and compute a preload responsiveness indicator using the obtained hemodynamic variable.

In some aspects, the techniques described herein relate to a system, wherein the waveform is obtained from at least one of a right ventricle, a right atrium, a pulmonary artery, and a pulmonary vein.

In some aspects, the techniques described herein relate to a system, wherein the waveform is obtained using a pulmonary artery catheter or a right ventricular catheter.

In some aspects, the techniques described herein relate to a system, wherein the waveform is a blood flow waveform measured from a right ventricle or a pulmonary artery.

In some aspects, the techniques described herein relate to a system, wherein the blood flow waveform is measured using an invasive ultrasound probe.

In some aspects, the techniques described herein relate to a system, wherein the waveform is a blood flow waveform computed from a right ventricular blood pressure waveform or a pulmonary artery blood pressure waveform.

In some aspects, the techniques described herein relate to a system, wherein the blood flow waveform is computed using a model of an input impedance of pulmonary circulation.

In some aspects, the techniques described herein relate to a system, wherein the model of the input impedance of the pulmonary circulation is based on a physiological Windkessel model.

In some aspects, the techniques described herein relate to a system, wherein the model of the input impedance of the pulmonary circulation is a machine learning model.

In some aspects, the techniques described herein relate to a system, wherein the waveform is obtained in real-time.

In some aspects, the techniques described herein relate to a system, wherein the waveform represents a time period of at least 5 seconds.

In some aspects, the techniques described herein relate to a system, wherein the instruction to obtain the waveform occurs on a continuous basis.

In some aspects, the techniques described herein relate to a system, wherein the obtained hemodynamic variable is selected from right ventricular stroke volume, right ventricular pulse pressure, right ventricular systolic pressure, pulmonary artery pulse pressure, pulmonary artery systolic pressure.

In some aspects, the techniques described herein relate to a system, wherein the obtained hemodynamic variable is right ventricular stroke volume, wherein right ventricular stroke volume is defined as a standard deviation of pulmonary arterial pressure of that beat.

In some aspects, the techniques described herein relate to a system, wherein the obtained hemodynamic variable is right ventricular stroke volume, wherein right ventricular stroke volume for each beat is defined as a standard deviation of pulmonary arterial pressure of that beat normalized by a mean pulmonary arterial pressure of that beat.

In some aspects, the techniques described herein relate to a system, wherein the instruction to obtain a hemodynamic variable includes an instruction to filter the hemodynamic variables.

In some aspects, the techniques described herein relate to a system, wherein the instruction to filter includes one or more instruction selected from remove beats whose values of the hemodynamic variable are at least two standard deviations greater than or at least two standard deviations below its mean, remove beats whose values of the hemodynamic variable are in a highest 5% of data, and remove beats whose values of the hemodynamic variables are in the lowest 5% of data.

In some aspects, the techniques described herein relate to a system, wherein the instruction to compute a preload responsiveness includes instructions to identify a maximum value (max) of the hemodynamic variable, identify a minimum value (min) of the hemodynamic variable, calculate a mean value (mean) of the max and the min, and compute the preload responsiveness indicator as

$$\frac{max - min}{mean} \times 100.$$

In some aspects, the techniques described herein relate to a system, wherein the max is a largest value of the hemodynamic variable.

In some aspects, the techniques described herein relate to a system, wherein the max is an average of at least two largest values of the hemodynamic variable.

In some aspects, the techniques described herein relate to a system, wherein the min is a smallest value of the hemodynamic variable.

In some aspects, the techniques described herein relate to a system, wherein the min is an average of at least two smallest values of the hemodynamic variable.

In some aspects, the techniques described herein relate to a system, wherein the instruction to compute a preload responsiveness includes instructions to identify a response pattern in the hemodynamic variable by bandpass filtering the hemodynamic variable, identify a maximum value (max) from the identified response pattern, identify a minimum value (min) from the identified response pattern, calculate a mean value (mean) of the max and the min, and compute the preload responsiveness indicator as

$$\frac{max - min}{mean} \times 100.$$

In some aspects, the techniques described herein relate to a system, wherein the max is a largest value of the response pattern.

In some aspects, the techniques described herein relate to a system, wherein the max is an average of at least two largest values of the response pattern.

In some aspects, the techniques described herein relate to a system, wherein the min is a smallest value of the response pattern.

In some aspects, the techniques described herein relate to a system, wherein the min is an average of at least two smallest values of the response pattern.

In some aspects, the techniques described herein relate to a system, wherein the instruction to compute a preload responsiveness includes instructions to identify a response pattern in the hemodynamic variable by bandpass filtering the hemodynamic variable, wherein the bandpass filtering possesses a frequency range, identify a modulation pattern in the waveform, wherein the modulation pattern is associated with a stimulus that affects blood pressure or blood flow, compute a power density spectrum of the hemodynamic variable from the identified heart beats in the modulation pattern, and compute the preload responsiveness indicator as a mean value (mean) of an amplitude of the power density spectrum over the frequency range.

In some aspects, the techniques described herein relate to a system, wherein the stimulus is selected from one or more of fluid infusion, vasopressor infusion, vasodilator infusion, spontaneous respiration, and mechanical respiration.

In some aspects, the techniques described herein relate to a system, wherein the frequency range is approximately 0.08 Hz to approximately 0.33 Hz.

In some aspects, the techniques described herein relate to a system, wherein the plurality of heart beats represents at least one respiratory cycle.

In some aspects, the techniques described herein relate to a system, wherein the memory further includes instructions to provide a medical intervention to the subject or provide directions to provide a medical intervention to the subject.

In some aspects, the techniques described herein relate to a system, wherein the medical intervention includes one or more of providing a fluid bolus, providing mechanical ventilation, and a pharmacological agent.

In some aspects, the techniques described herein relate to a system, wherein the pharmacological agent is selected from a vasopressor and a vasodilator.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1A illustrates an example of a waveform P(t) of blood pressure taken over a single heart cycle.

FIG. 1B illustrates exemplary waveform morphology obtained from a pulmonary artery.

FIG. 1C illustrates a waveform morphology obtained from a right ventricle.

FIG. 2 illustrates an example of how analog signals can be digitized into a sequence of digital values using any standard analog-to-digital converter.

FIG. 3 illustrates an example of a blood pressure waveform comprised of individual beats.

FIG. 4 illustrates a flow chart of a method to determine preload responsiveness for a patient.

FIG. 5 illustrates a schematic of an exemplary computing device.

FIG. 6 illustrates a distributed computing device.

FIG. 7 illustrates a hemodynamic monitoring system

### DETAILED DESCRIPTION

The current disclosure details systems and methods to evaluate stroke volume variation (SVV) utilizing hemodynamic data that is derived from a continuous blood pressure sensor, and especially from right-side heart parameters. SVV can be used as an indicator of preload responsiveness. Preload responsiveness indicates how an increase in end diastolic volume results in an increased stroke volume. Preload responsiveness can indicate how well an individual responds to an external stimulus, including (but not limited to) fluid response, drug response, mechanical ventilation (or respiration), and/or any other stimulus. Under normal conditions, most subjects are preload responsive over the normal range of right ventricular end-diastolic volume and left ventricular end-diastolic volume.

Current methods to determine SVV and other parameters use measurements of the arterial side (i.e., “left-side”) of the heart. However, due to physiological phenomena, arterial side measurements lack sensitivity and/or are susceptible to physiological modulation that can obscure variation. Thus, a right-side (e.g., pulmonic, venal, etc.) system to measure and determine many parameters is desirable. In some implementations, a sensor within the right-side of the heart is utilized to sense right-side blood pressure. In some implementations, a sensor in another location (i.e., not right side) is utilized to compute right-side blood pressure. Continuous sensing can be utilized to derive a right-side blood pressure waveform.

A right-side blood pressure waveform can be utilized to derive SVV estimates based on measurements of systemic “right side” (e.g., pulmonic, right ventricular, venal) blood pressure. Various systems and methods of the disclosure determine beat-to-beat of SVV, which can be determined as a function of the standard deviation of the blood pressure waveform over a plurality of cardiac cycles. Other hemodynamic parameters can be derived from SVV, or determined from waveform concurrently with determining SVV.

The various systems and methods may be used with any type animal subject, especially human. The various systems and methods may be used in a variety of environments in which continuous blood pressure monitoring can be accomplished. Accordingly, it is to be understood that the use of term “patient” should not be limited to any specific animal or any specific environment, but inclusive any animal and any environment that allows for continuous blood pressure monitoring.

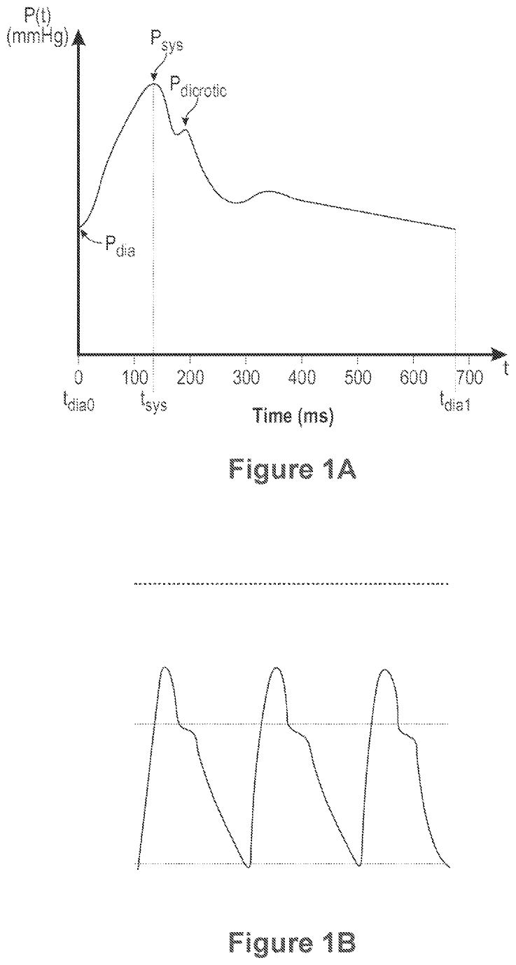

FIG. 1A provides an example of a waveform P(t) of blood pressure taken over a single heart cycle (also referred to as a cardiac cycle, heartbeat, or beat). Specifically, FIG. 1A illustrates a waveform that can be defined by its hemodynamic parameters (also referred to as hemodynamic variables): from the point of diastolic pressure P_{dia} at time t_{dia0}, through the time t_{sys} of systolic pressure P_{sys}, to a time t_{dia1} at which the blood pressure once again reaches P_{dia}. Additional hemodynamic parameters (or variables) can be defined based on or derived from these measurements. For example, pulse pressure (PP) can be defined as the difference between P_{sys} and P_{dia}—e.g., if P_{sys}=120 mmHg and P_{dia}=80 mmHg, then PP=40 mmHg. Additionally, a mean pressure can be defined as the area under the curve (i.e., single waveform) divided by the cardiac cycle time (e.g., t_{dia1}−t_{dia0}). Mean pressure can be mean arterial pressure (MAP), mean ventricular pressure (MVP), or any other mean pressure that is relevant to the location being measured (e.g., capillary, atrial, venal, etc.).

In many implementations, P(t), or any signal that is proportional to P(t), is obtained as a right-side measurement, which can be obtained with a blood pressure sensor or blood flow sensor either invasively or non-invasively. Placement of non-invasive sensor will typically be dictated by the instruments themselves—for example, blood pressure cuffs and clamps can be a finger cuff, an upper arm cuff, a toe cuff, and an earlobe clamp. An invasive instruments such as a catheter-mounted pressure transducers can be place in any applicable location for measuring blood pressure, such as intraarterial and intracardiac pressure transducers. In some implementations, right-side blood pressure measurements are obtained invasively directly from right ventricle of the heart and/or pulmonary artery. Various pulmonary artery and right heart catheters can be utilized, such as (for example) a Swan-Ganz catheter, which may be preferable due to multiple transducers to obtain ventricular pressure and pulmonary arterial pressure simultaneously.

The singular waveform illustrated in FIG. 1A is meant to be illustrative of one waveform shape, and blood pressure waveforms can vary in shape and/or morphology depending on where such waveforms are obtained. For example, FIG. 1B illustrates exemplary waveform morphology obtained from a pulmonary artery, while FIG. 1C illustrates a waveform morphology obtained from a right ventricle. One of ordinary skill in the art will understand the various landmarks identified in FIG. 1A apply to waveform morphologies obtained from different locations. While FIGS. 1A-1C provide waveforms obtained from blood pressure sensors, waveforms can be obtained from a blood flow sensor, either separate from or in conjunction with a blood pressure sensor.

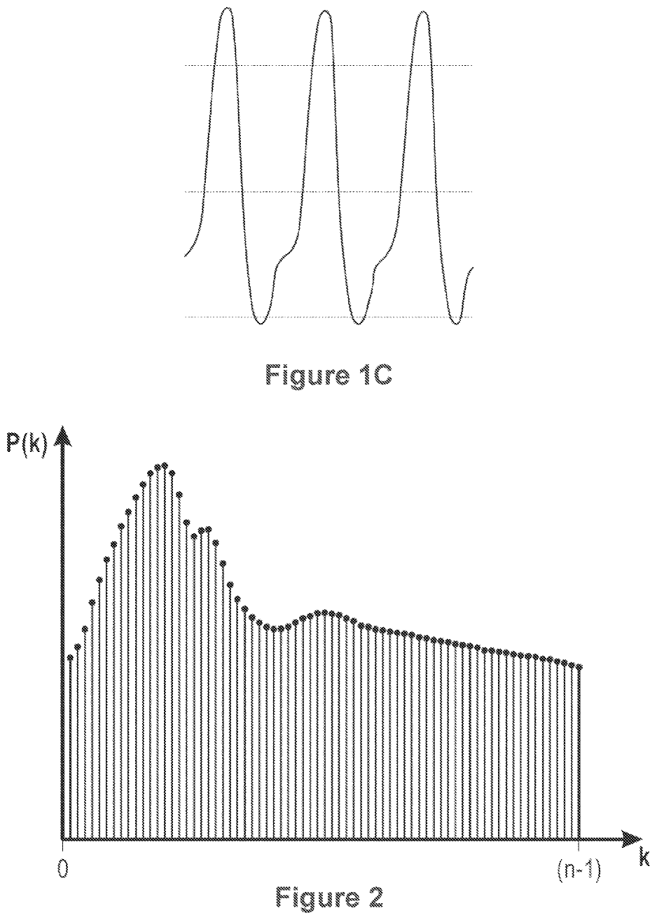

As is known, and as is illustrated in FIG. 2, analog signals of pressure waveforms (e.g., P(t)) can be digitized into a sequence of digital values using any standard analog-to-digital converter (ADC). For example, P(t), t0≤t≤tf, can be converted into the digital form P (k), k=0, (n−1), where t₀ and t_{f} are initial and final times, respectively, of the computation interval and n is the number of samples of P(t) to be included in the calculations, distributed usually evenly over the computation interval.

The pressure values illustrated in FIG. 2 allow for statistical analyses of the values, which can be performed utilizing one or more beats (or a fraction of a beat). The values can be utilized to compute various parameters, such as (for example) mean pressure, median pressure, maximum pressure (e.g., systolic pressure), minimum pressure (e.g., diastolic pressure), standard deviation (e.g., within one beat or across multiple beats), variance (e.g., within one beat or across multiple beats), slope (e.g., of rising side, of falling side, etc.), shape function (e.g., pattern of a curve), and percentiles (including quartiles, quintiles, deciles, etc.) of pressure. Other statistical measurements can also be computed from the plurality of values.

The mean and standard deviation of a continuous or discrete function or data set f can be calculated, and is represented in equations by the Greek letters μ and σ, respectively, or in programming languages, by function names such as mean and std. Thus, μ(f) and mean(f) represent the mean of the function or data set f and σ(f) and std(f) represent its standard deviation.

While FIGS. 1A-1C and FIG. 2 provide examples blood pressure waveforms, blood flow waveforms captured by blood flow sensors or derived from a blood pressure waveform. Such derivations to generate blood flow waveforms can be generated de novo or using known methods. In some instances, a model of input impedance of pulmonary circulation is used to compute a blood flow waveform—an exemplary model is the physiological Windkessel model. Alternatively, a machine learning model can be trained to generate a blood flow waveform.

As noted previously, preload responsiveness is important to determine the adequacy of resuscitation therapy (e.g., fluid, pharmacological, etc.) and to improve cardiac performance and organ perfusion. SVV can be used as an indicator of preload responsiveness and can be utilized to determine types of interventions, including a fluid bolus, mechanical respiration, and/or pharmacological intervention (e.g., vasopressors or vasodilators). Thus, an accurate determination of SVV is important for determining proper care of a patient.

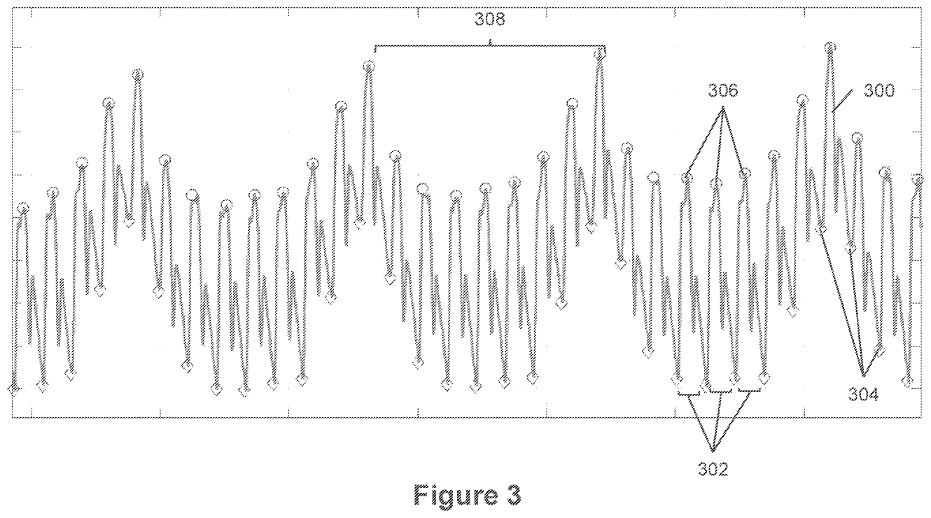

FIG. 3 provides an illustration of right-heart pressure waveform comprising multiple cardiac cycles (i.e., beats). As illustrated, blood pressure waveform 300 is comprised of individual beats 302, which are identifiable as time between diastolic pressures 304 (minima for each beat 302). Each beat 302 is further characterizable by a systolic pressure 306. While the start and end of an individual beat can be defined as time between diastolic pressures, the start and end of a beat can instead be defined as time between any other cyclic feature (e.g., time between systolic pressures, time between dicrotic notches, etc.).

Also shown are respiratory cycles 308, which can be identified as starting and ending between two beats having maximum diastolic pressures. Respiratory cycles 308 can cause variation in volume and pressure, which introduce additional variation and noise into the collected waveforms, which may cause inaccuracies in SVV determination. In some implementations, once beats are identified, certain beats can be filtered from the waveform, to allow for a more accurate SVV determination. Filtering can remove beats based on statistical analyses and/or arithmetic analyses. For example, beats can be filtered out if they fit one or more of the following categories: beats that are statistical outliers; beats that are a certain number of standard deviations away from a mean (e.g., 1 std, 1.5 std, 2 std, 3 std, etc.); beats within the upper and/or lower extremities of data (e.g., top 1%, top 2%, top 3%, top 4%, top 5%, top 10%, bottom 1%, bottom 2%, bottom 3%, bottom 4%, bottom 5%, bottom 10%, etc.); and/or any other metric that can filter beat waveforms to limit noise in the collected waveform. In some implementations, beat data are smoothed.

Once beats have been identified and optionally filtered and/or smoothed, SVV can be determined from the right-heart waveform. As SVV is a derived parameter, various equations can be implemented to compute SVV. An exemplary equation can include the following:

$$SVV = 100 * \frac{max - min}{( max + min ) / 2} \qquad ( eq . 1 )$$

where the maximum (max) and minimum (min) are parameter based on one or more beats.

The maximum and minimum can be selected as the highest (or largest) and lowest (or smallest) value, by pressure (e.g., systolic pressure, average pressure, MAP, MVP, standard deviation, diastolic pressure, etc.). As blood pressure waveform recordings can comprise multiple beats. Maximum and/or minimum can also be selected from multiple beats (e.g., an average parameter between 2 beats, 3 beats, 4 beats, etc.). Standard deviation can be selected of standard deviation within a single beat or across multiple beats (e.g., 2 beats, 3 beats, 4 beats, etc.). Additionally, as blood pressure waveform measurements (e.g., as illustrated in FIG. 3) include multiple beats and possibly multiple respiratory cycles, the maximum and/or minimum parameters can come from neighboring (i.e., consecutive) beats and/or from non-neighboring beats. For example, the maximum can be the standard deviation derived from two beats with the highest pressure, while minimum can be standard deviation derived from two beats with the lowest pressure (where the pressure can be systolic pressure, diastolic pressure, average pressure, etc.).

In some implementations, minimum and/or maximum parameters can be normalized. Normalization can include various modifiers to a maximum and/or minimum, such as addition, subtraction, division, multiplication, function (e.g., a mathematical function), and/or any other methodology for normalizing a value. In some implementations, the normalization can be relative to another parameter from the values, such as systolic pressure, dicrotic notch pressure, minimum pressure, mean pressure (e.g., MAP, MVP, etc.). In some implementations, if a value is obtained across multiple beats, normalizing values can be an average of the same beats. For example, if maximum is a standard deviation of the two, highest pressure beats, normalizing can comprise dividing that value by the average mean pressure between the two beats. In such implementations, standard deviation of the two highest pressure beats are divided by the average mean pressure between these two beats and standard deviation of the two lowest pressure beats are divided by the average mean pressure between these two beats. Once modified minimum and maximum values are generated, SVV can be computed via an equation, such as illustrated in Equation 1.

While the above describes a computation of SVV, additional metrics of variation can be computed using such waveforms, including pulse pressure variation (PPV) and/or systolic pressure variation (SPV). As described above, PP can be defined as the difference between P_{sys} and P_{dia}. Computation of PPV and/or SPV can follow equations similar to Equation 1, for example:

$$PPV = 100 * \frac{max PP - min PP}{( max PP + min PP ) / 2} \qquad ( eq . 2 )$$

$$SPV = 100 * \frac{max Sys - min Sys}{( max Sys + min Sys ) / 2} \qquad ( eq . 3 )$$

where maxPP is a maximum pulse pressure, minPP is a minimum pulse pressure, maxSys is a maximum systolic pressure, and minSys is a minimum systolic pressure. Such computations of PPV and/or SPV can include filtering, combining, averaging, and/or any other mathematical calculation to generate values for maxPP, minPP, maxSys, and/or minSys, such as described herein.

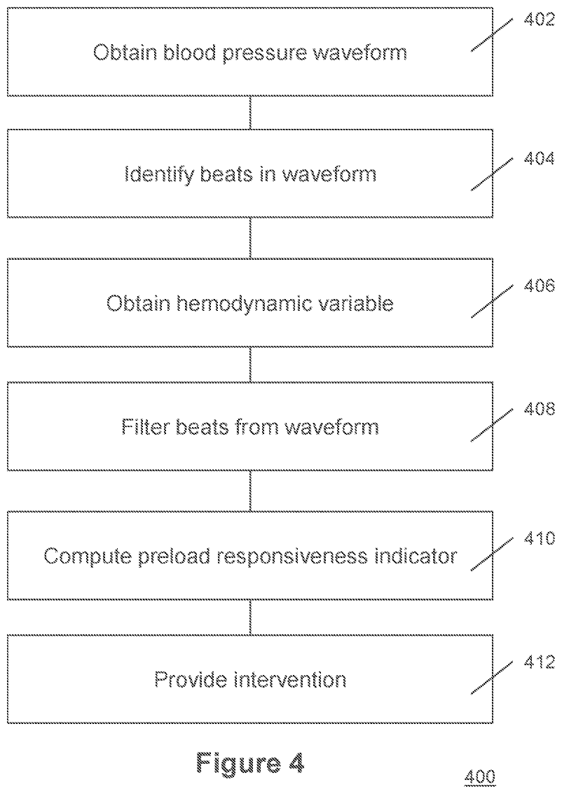

Several systems and methods of the disclosure provide for a practical application of determining fluid responsiveness of an individual. Such systems and methods can utilize or follow process 400 illustrated in FIG. 4. Process 400 is an exemplary process that can be used to assess preload responsiveness for a patient and optionally provide a medical intervention (e.g., treatment) for the individual based on the preload responsiveness assessment.

In process 400, a blood pressure or blood flow waveform is obtained for an individual at 402. In many instances, the waveform is obtained from a right heart or pulmonary artery. Such waveforms can be obtained live and/or in real-time, such as when the individual is currently under a medical procedure and/or clinical monitoring, and/or as a trace file from a previously obtained (e.g., a recording) blood pressure waveform. Waveforms, either live/real-time or recorded, can be from any applicable location or methodology to obtain such waveforms, as described herein. In some instances, blood pressure or blood flow is acquired a continual or continuous basis to yield a waveform

Waveforms can be generated from blood pressure, blood flow, and/or any other measurable cardiac parameter. As mentioned previously, certain benefits apply to right-side measurements, thus certain implementations generate a waveform form in one or more of the following locations: right ventricle, right atrium, pulmonary artery, and pulmonary vein, which can be derived or directly sensed. Waveform traces can be generated from multiple locations simultaneously, such as from a right ventricle and pulmonary artery. Directly sensed measurements can be obtained from an appropriate catheter, such as a pulmonary artery catheter and/or a right ventricular catheter. A Swan-Ganz catheter is capable of sensing right ventricle and pulmonary artery pressures simultaneously via multiple pressure transducers that can be simultaneously situated within the right ventricle and pulmonary artery. An ultrasound probe can also be used to obtain a waveform. The right-heart catheter can include a port located within a tip portion of the right-heart catheter. The tip portion can be positioned within a pulmonary artery of a patient (such as by insertion through jugular vein). A pressure or flow sensor configured to sense a right-heart blood pressure or flow of the patient through a lumen in communication with the port.

At 404, beats can be identified within the blood pressure waveform. Beat identification can be performed a variety of ways, such as identifying cyclical features within the waveforms, such as local minima (e.g., diastolic pressures), maxima (e.g., systolic pressures), dicrotic notches, and/or any other waveform landmark that can indicate a cardiac cycle or beat.

At 406, one or more hemodynamic variables (or parameters) for each beat in the waveform can be obtained and/or identified. As noted herein, such variables can be obtained from a waveform, such as described herein, including maximum pressure, minimum pressure, systolic pressure, diastolic pressure, stroke volume, etc. Such variables can be obtained from one or more locations, such as right ventricle and/or pulmonary artery, such that right ventricular systolic pressure and pulmonary artery systolic pressure may both be utilized. Some hemodynamic variables are determined as a computation or calculation of other hemodynamic variables. For example, stroke volume for a beat can be defined as proportional to a standard deviation of arterial pressure of that beat. The standard deviation can further be normalized by a mean arterial pressure for that beat. As a specific example, right ventricular stroke volume can be defined as proportional to a standard deviation of pulmonary arterial pressure of that beat-optionally, the standard deviation can be normalized by a mean arterial pressure.

A hemodynamic variable can be obtained from each beat in the waveform or as a metric across multiple heartbeats. Multiple heartbeats can be defined by a pattern within the waveform (e.g., a respiratory cycle) or as a period of time (e.g., 1 second, 2 seconds, 3 seconds, 4 seconds, 5 seconds, 10 seconds, 15 seconds, etc.)

Filtering and/or smoothing can optionally be performed at 408 to remove noise from the waveforms. Such filtering can be performed using windows that can defined by an amount of time or a number of beats (e.g., as identified in 404). Time-based windows can be defined as, for example, approximately 5 seconds, 10 seconds, 15 seconds, 20 seconds, 30 seconds, 45 seconds, 60 seconds, or more, where approximately is ±25% or up to 10 seconds, depending on amount of time utilized. Beat-based windows can defined as a number of beats, such as approximately 5 beats, 10 beats, 15 beats, 20 beats, 25 beats, 30 beats, 35 beats, 40 beats, 45 beats, 50 beats, 75 beats, 100 beats, where approximately is ±25% or up to 10 beats, depending on the number of beats utilized. Windows can be discrete windows or sliding windows. For example, 20-second discrete windows, exemplary windows would comprise windows from 0 seconds to 20 seconds, 20 seconds to 40 seconds, 40 seconds to 60 seconds, etc., while 20-second sliding windows would comprise windows covering time periods 0-20 seconds, 1-21 seconds, 2-22 seconds, etc. When performing filtering in real time, a window can be the immediately prior set of time (e.g., the last 20 seconds) or the immediately prior set of beats (e.g., the last 20 beats).

In one filtering method, beats with a hemodynamic variable that is equal to and/or more than a number of standard deviations from the mean may be excluded—for example, beats can be removed when the hemodynamic variable is at least two standard deviations greater than the mean and/or two standard deviations below the mean. It should be noted that two standard deviations is merely an example, and the threshold can be set to any number of standard deviations to remove aberrant and/or outlier data, such as 1 standard deviation, 1.5 standard deviations, 2 standard deviations, 2.5 standard deviations, 3 standard deviations or more. Another filtering method, which can be used in lieu of or in addition to standard deviation filtering, is to remove beats that are in the extremities of data. For example, beats with a hemodynamic variable that is in the highest 5% and/or lowest 5% of data may be excluded. The 5% number is merely an example, and different percentages can be used, including 1%, 2%, 3%, 4%, 5%, 6%, 7%, 8%, 9%, 10%, 12.5%, 15%, 17.5%, 20%, 25%, etc. Different thresholds may be utilized for upper and lower limits and/or different parameters can be utilized on the upper and lower limits. For example, beats that are 2 standard deviations above a mean and beats in the lower 5% of data may be removed. Another example can exclude beats in the upper 5% of data and lower 2% of data. Other combinations and/or thresholds can be utilized to tune or filter data as appropriate for a specific use.

A preload responsiveness indicator can be computed at 410. As noted herein, preload response indicates how an increase in end diastolic volume results in an increased stroke volume. Preload responsiveness can inform how a patient will respond to an external stimulus, including (but not limited to) fluid response, drug response, and/or any other stimulus. One example of a preload responsiveness indicator is SVV, which can be computed for a patient based on the beats in the waveform. The computation can be accomplished as described herein, including using Equation 1. As mentioned, the minimum and maximum values can be from a single beat or from an averaged value from multiple beats. The minimum and maximum values can also be a statistical value such as a standard deviation. Such values can also be normalized, as described herein, including by dividing a standard deviation by the MAP or MVP as appropriate.

In some instances, computing a preload responsiveness indicator 410 can include identifying a response pattern in the waveform. Such instances may identify a response pattern by bandpass filtering the hemodynamic variable. Bandpass filtering can filter for beats within a specific frequency and/or within a specific range of the hemodynamic variable (e.g., removing beats that have too high and/or too low of a frequency and/or removing beats where the hemodynamic variable is too high and/or too low). In certain instances, the frequency range is approximately 0.08 Hz to approximately 0.33 Hz (where approximately indicates ±10% and/or ±0.15 Hz). Maximum and/or minimum values can be identified from the response pattern. As mentioned, the minimum and maximum values can be from a single beat or from an averaged value from multiple beats within the response pattern. The computation can be accomplished as described herein, including using Equation 1. The minimum and maximum values can also be a statistical value such as a standard deviation. Such values can also be normalized, as described herein, including by dividing a standard deviation by the MAP or MVP as appropriate.

In some instances, a modulation pattern is identified in a waveform. Such modulation pattern can follow identification of a response pattern, such as described herein, including by bandpass filtering for a particular frequency range. A modulation pattern can be identified by identifying a response (e.g., change in blood pressure and/or blood flow) to a stimulus, including (but not limited to) a fluid bolus, respiration (e.g., spontaneous and/or mechanical), and/or a dose of a pharmacological agent (e.g., vasopressor and/or vasodilator). A power density spectrum of the hemodynamic variable can be computed from the identified beats within a modulation pattern. The preload responsiveness indicator can then be computed as a mean value (mean) of an amplitude of the power density spectrum over a frequency range (e.g., the frequency range from bandpass filtering).

Preload responsiveness can be utilized to assess the appropriateness of resuscitation therapy to improve optimal cardiac performance and organ perfusion. Thus at 412, a patient can be provided a medical intervention based on the determined preload responsiveness. For example, if a person's blood pressure is declining and/or falls below a certain threshold, a fluid bolus can be provided to the person, if they have an adequate fluid responsiveness. Alternatively, a pharmacological agent (e.g., vasopressor and/or vasodilator) can be provided to the individual, if the fluid responsiveness is minimal. Additionally, mechanical respiration (or mechanical ventilation) can be provided in certain circumstances. In some instances, the intervention can be provided automatically by a medical controller, health monitor, and/or other computing device, while other instances may provide an instruction to provide the intervention. In some instances, the intervention (either automatic or instructed) can include additional information such as volume, dose, infusion rate, and/or any other relevant information for the intervention.

It should be noted that while process 400 is described specific to preload responsiveness, this process can be modified to compute PPV and/or SPV as a proxy or indicator of another cardiac parameter that can guide medical intervention. As will be appreciated, PPV and/or SPV can be computed using these procedures and the applicable Equation 2 and/or Equation 3, as appropriate.

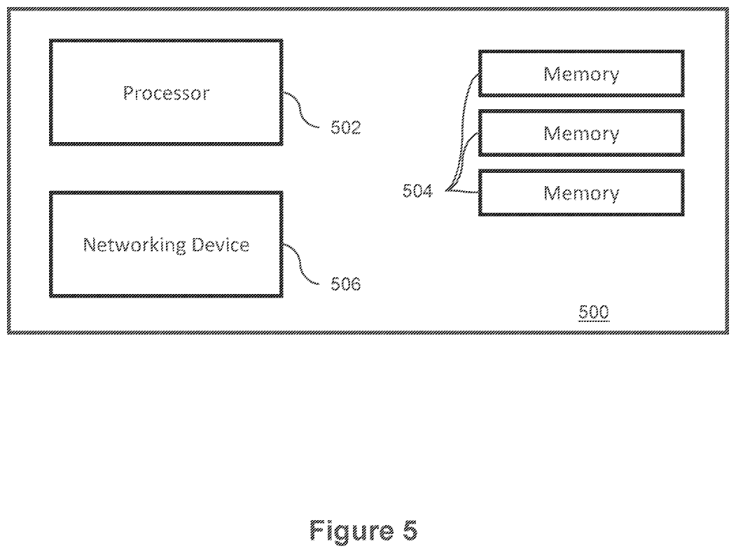

Processes that provide the methods and systems for determining cardiovascular parameters in accordance with some implementations are executed by a computing device or computing system, such as a desktop computer, tablet, mobile device, laptop computer, notebook computer, server system, and/or any other device capable of performing one or more features, functions, methods, and/or steps as described herein. The relevant components in a computing device that can perform the processes are shown in FIG. 5. One skilled in the art will recognize that computing devices or systems may include other components that are omitted for brevity without departing from described implementations. A computing device 500 can comprises a processor 502 and at least one memory 504. Memory 504 can be a non-volatile memory and/or a volatile memory, and the processor 502 is a processor, microprocessor, controller, or a combination of processors, microprocessor, and/or controllers that performs instructions stored in memory 504. Such instructions stored in the memory 504, when executed by the processor, can direct the processor, to perform one or more features, functions, methods, and/or steps as described herein. Any input information or data can be stored in the memory 504—either the same memory or another memory. The computing device 500 may have hardware and/or firmware that can include the instructions and/or perform these processes.

Certain computing devices can include a networking device 506 to allow communication (wired, wireless, etc.) to another device, such as through a network, near-field communication, Bluetooth, infrared, radio frequency, and/or any other suitable communication system. Such systems can be beneficial for receiving data, information, or input (e.g., images) from another computing device and/or for transmitting data, information, or output (e.g., quality score, rating, etc.) to another device. The networking device can be used to send and/or receive update models, interfaces, etc. to a user device.

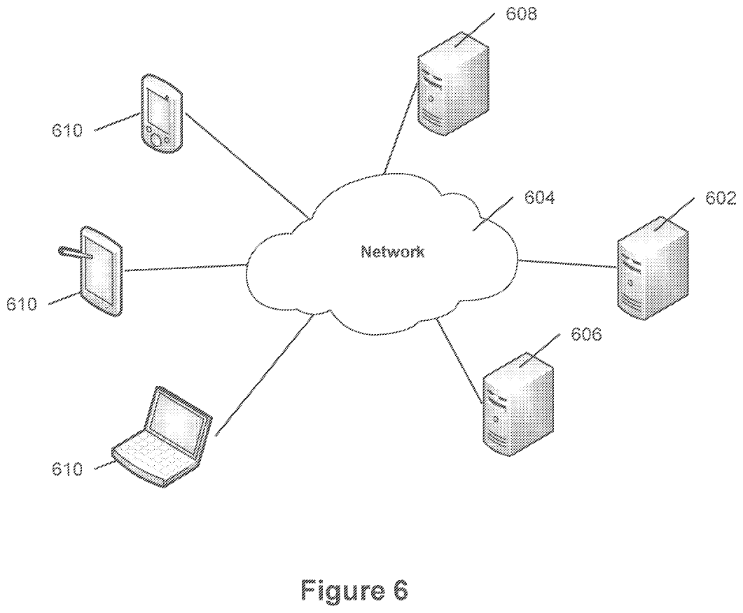

Turning to FIG. 6, a distributed computing device that can be utilized is illustrated. Distributed computing devices may be useful where computing power is not possible at a local level, and a central computing device (e.g., server) performs one or more features, functions, methods, and/or steps described herein. A computing device 602 (e.g., server) can be connected to a network 604 (wired and/or wireless), where it can receive inputs from one or more computing devices, including data from a records database or repository 606, data provided from a laboratory computing device 608, and/or any other relevant information from one or more other remote devices 610. Once computing device 602 performs one or more features, functions, methods, and/or steps described herein, any outputs can be transmitted to one or more computing devices 606, 608, 610 for entering into records.

In additional implementations, the instructions for the processes can be stored in any of a variety of non-transitory computer readable media appropriate to a specific application.

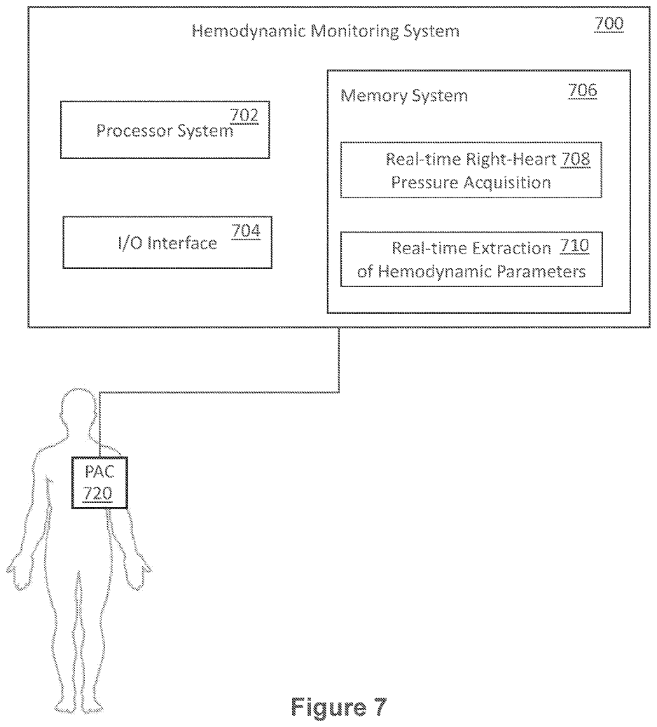

The systems and methods of the current disclosure can be utilized within or performed using a hemodynamic monitoring system. In some instances, the hemodynamic monitoring system includes a right-heart sensor, such as a right ventricle catheter or a pulmonary artery catheter (PAC; e.g., Swan-Ganz catheter). Provided in FIG. 7 is an example of a hemodynamic monitoring system 700 as would be utilized to measure right-heart blood pressure of a patient. Within the patient is a PAC 720 (or other right-heart catheter) that may comprise one or more sensors such as (for example) a pressure sensor, thermal sensor (e.g., for measurement of cardiac output via thermodilution), and a fiber optics (e.g., photometric or other optical measurements). PAC 720 can be in communication with hemodynamic monitoring system 700.

Hemodynamic monitoring system 700 can comprise a computational system, such as (for example) the system portrayed and described in reference to FIG. 5. Hemodynamic monitoring system 700 can comprise a processor system 702 and I/O interface 704 for input and output of data, such as data communicated between hemodynamic monitoring system 700, a sensor of PAC 720, and a user interface. Hemodynamic monitoring system 700 can utilize a number of applications stored within a memory system 706 to be executed by processor system 702. Applications that can be stored within a memory system 706 include real-time right-heart pressure acquisition applications 708, and real-time extraction of hemodynamic parameters 710, which can be displayed via the hemodynamic monitoring system.

### Doctrine of Equivalents

Having described several implementations, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred implementations of the present invention are merely illustrative of the invention as a whole, and that variations in the components or steps of the present invention may be made within the spirit and scope of the invention. Accordingly, the present invention is not limited to the specific implementations described herein, but, rather, is defined by the scope of the appended claims.

### Examples

Example 1. A real-time method for computing a preload responsiveness indicator, comprising:
generating, using a hemodynamic monitoring system, a waveform from a patient's right heart circulation in real-time, wherein the waveform comprises a plurality of heart beats, and wherein the waveform is a blood pressure waveform or a blood flow waveform;
identifying, using the hemodynamic monitoring system, individual heart beats in the waveform;
obtaining, using the hemodynamic monitoring system, a hemodynamic variable for each identified heart beat in the waveform; and
computing, using the hemodynamic monitoring system, a preload responsiveness indicator using the obtained hemodynamic variable.

Example 2. The method of example 1, wherein generating the waveform comprises:
sensing blood pressure or blood flow from at least one of: a right ventricle, a right atrium, a pulmonary artery, and a pulmonary vein; or
deriving blood pressure or blood flow representative of: a right ventricle, a right atrium, a pulmonary artery, and a pulmonary vein.

Example 3. The method of example 2, wherein sensing blood pressure or blood flow comprises the use a pulmonary artery catheter or a right ventricular catheter.

Example 4. The method of any one of examples 1-3, wherein the waveform is a blood flow waveform of a right ventricle or a pulmonary artery.

Example 5. The method of example 4, wherein the blood flow waveform is measured using an invasive ultrasound probe.

Example 6. The method of any one of examples 1-3, wherein the waveform is a blood flow waveform computed from a right ventricular blood pressure waveform or a pulmonary artery blood pressure waveform.

Example 7. The method of example 6, wherein the blood flow waveform is computed using a model of an input impedance of pulmonary circulation.

Example 8. The method of example 7, wherein the model of the input impedance of the pulmonary circulation is based on a physiological Windkessel model.

Example 9. The method of example 7, wherein the model of the input impedance of the pulmonary circulation is a machine learning model.

Example 10. The method of any one of examples 1-9, wherein the hemodynamic variable is selected from the group consisting of: right ventricular stroke volume, right ventricular pulse pressure, right ventricular systolic pressure, pulmonary artery pulse pressure, and pulmonary artery systolic pressure.

Example 11. The method of example 10, wherein the hemodynamic variable is right ventricular stroke volume, wherein right ventricular stroke volume is defined as proportional to a standard deviation of pulmonary arterial pressure of that beat.

Example 12. The method of example 10, wherein the hemodynamic variable is right ventricular stroke volume, wherein right ventricular stroke volume for each beat is defined as proportional to a standard deviation of pulmonary arterial pressure of that beat normalized by a mean pulmonary arterial pressure of that beat.

Example 13. The method of anyone of example 1-12, wherein obtaining a hemodynamic variable comprises filtering the hemodynamic variables.

Example 14. The method of example 13, wherein filtering comprises one or more of:
removing beats whose values of the hemodynamic variable are at least two standard deviations greater than or at least two standard deviations below its mean;
removing beats whose values of the hemodynamic variable are in a highest 5% of data; or removing beats whose values of the hemodynamic variables are in a lowest 5% of data.

Example 15. The method of any one of examples 1-14, wherein the preload responsiveness indicator is stroke volume variation, wherein computing a preload responsiveness indicator comprises:
identifying a maximum value (max) of the hemodynamic variable;
identifying a minimum value (min) of the hemodynamic variable;
calculating a mean value (mean) of the max and the min; and
computing the preload responsiveness indicator as: (max−min)/mean×100.

Example 16. The method of example 15, wherein the max is a largest value of the hemodynamic variable.

Example 17. The method of example 15, wherein the max is an average of at least two largest values of the hemodynamic variable.

Example 18. The method of example 15, wherein the min is a smallest value of the hemodynamic variable.

Example 19. The method of example 15, wherein the min is an average of at least two smallest values of the hemodynamic variable.

Example 20. The method of any one of examples 1-14, wherein the preload responsiveness indicator is stroke volume variation, wherein computing a preload responsiveness comprises:
identifying a response pattern in the hemodynamic variable by bandpass filtering the hemodynamic variable;
identifying a maximum value (max) from the identified response pattern;
identifying a minimum value (min) from the identified response pattern;
calculating a mean value (mean) of the max and the min; and
computing the preload responsiveness indicator as: (max−min)/mean×100.

Example 21. The method of example 20, wherein the max is a largest value of the response pattern.

Example 22. The method of example 20, wherein the max is an average of at least two largest values of the response pattern.

Example 23. The method of example 20, wherein the min is a smallest value of the response pattern.

Example 24. The method of example 20, wherein the min is an average of at least two smallest values of the response pattern.

Example 25. The method of any one of examples 1-14, wherein computing a preload responsiveness comprises:
identifying a response pattern in the hemodynamic variable by bandpass filtering the hemodynamic variable, wherein the bandpass filtering possesses a frequency range;
identifying a modulation pattern in the waveform, wherein the modulation pattern is associated with a stimulus that affects blood pressure or blood flow;
computing a power density spectrum of the hemodynamic variable from the identified heart beats in the modulation pattern; and
computing the preload responsiveness indicator as a mean value (mean) of an amplitude of the power density spectrum over the frequency range.

Example 26. The method of example 25, wherein the stimulus is selected from one or more of the group consisting of: fluid infusion, vasopressor infusion, vasodilator infusion, spontaneous respiration, and mechanical respiration.

Example 27. The method of example 25 or 26, wherein the frequency range is approximately 0.08 Hz to approximately 0.33 Hz.

Example 28. The method of any one of examples 1-27, wherein the plurality of heart beats represents at least one respiratory cycle.

Example 29. The method of any one of examples 1-28 further comprising providing, using the hemodynamic monitoring system, directions to perform a medical intervention to the patient.

Example 30. The method of any one of examples 1-29 further comprising performing a medical intervention to the patient.

Example 31. The method of 30, wherein performing a medical intervention to a patient is automated by the hemodynamic monitoring system.

Example 32. The method of any one of examples 29-31, wherein the medical intervention comprises one or more of: administering a fluid bolus, providing mechanical ventilation, or administering a pharmacological agent.

Example 33. The method of example 32, wherein the pharmacological agent is selected from a vasopressor and a vasodilator.

Example 34. A hemodynamic monitoring system for determining a preload responsiveness indicator, comprising:
a pressure or flow sensor configured to sense a right-heart blood pressure or flow of a patient; and
a computational system in communication with the pressure or flow sensor, the computational system comprising at least one processor and a memory comprising instructions that when executed instruct the computational system to:
generate a waveform from a patient's right heart circulation in real-time, wherein the waveform comprises a plurality of heart beats, and wherein the waveform is a blood pressure waveform or a blood flow waveform;
identify individual heart beats in the waveform;
obtain a hemodynamic variable for each identified heart beat in the waveform; and
compute a preload responsiveness indicator using the obtained hemodynamic variable.

Example 35. The system of example 34, wherein the instruction to generate the waveform comprises an instruction to:
sense blood pressure or blood flow from at least one of: a right ventricle, a right atrium, a pulmonary artery, and a pulmonary vein; or
derive blood pressure or blood flow representative of: a right ventricle, a right atrium, a pulmonary artery, and a pulmonary vein.

Example 36. The system of example 35, wherein sensing blood pressure or blood flow comprises the use the pressure or flow sensor.

Example 37. The system of any one of examples 34-36, wherein the waveform is a blood flow waveform of a right ventricle or a pulmonary artery.

Example 38. The system of example 37, wherein the pressure or flow sensor is an invasive ultrasound probe to sense blood flow.

Example 39. The system of any one of example 34-36, wherein the waveform is a blood flow waveform computed from a right ventricular blood pressure waveform or a pulmonary artery blood pressure waveform.

Example 40. The system of example 39, wherein the blood flow waveform is computed using a model of an input impedance of pulmonary circulation.

Example 41. The system of example 40, wherein the model of the input impedance of the pulmonary circulation is based on a physiological Windkessel model.

Example 42. The system of any one of examples 34-41, wherein the hemodynamic variable is selected from the group consisting of: right ventricular stroke volume, right ventricular pulse pressure, right ventricular systolic pressure, pulmonary artery pulse pressure, and pulmonary artery systolic pressure.

Example 43. The system of example 43, wherein the obtained hemodynamic variable is right ventricular stroke volume, wherein right ventricular stroke volume is defined as a standard deviation of pulmonary arterial pressure of that beat.

Example 44. The system of example 43, wherein the hemodynamic variable is right ventricular stroke volume, wherein right ventricular stroke volume for each beat is defined as a standard deviation of pulmonary arterial pressure of that beat normalized by a mean pulmonary arterial pressure of that beat.

Example 45. The system of any one of examples 34-44, wherein the instruction to obtain a hemodynamic variable comprises an instruction to filter the hemodynamic variables.

Example 46. The system of example 45, wherein the instruction to filter comprises one or more instruction selected from:
remove beats whose values of the hemodynamic variable are at least two standard deviations greater than or at least two standard deviations below its mean;
remove beats whose values of the hemodynamic variable are in a highest 5% of data; or remove beats whose values of the hemodynamic variables are in a lowest 5% of data.

Example 47. The system of any one of examples 34-46, wherein the preload responsiveness indicator is stroke volume variation, wherein the instruction to compute a preload responsiveness comprises instructions to:
identify a maximum value (max) of the hemodynamic variable;
identify a minimum value (min) of the hemodynamic variable;
calculate a mean value (mean) of the max and the min; and
compute the preload responsiveness indicator as: (max−min)/mean×100.

Example 48. The system of example 47, wherein the max is a largest value of the hemodynamic variable.

Example 49. The system of example 47, wherein the max is an average of at least two largest values of the hemodynamic variable.

Example 50. The system of example 47, wherein the min is a smallest value of the hemodynamic variable.

Example 51. The system of example 47, wherein the min is an average of at least two smallest values of the hemodynamic variable.

Example 52. The system of any one of examples 34-51, wherein the preload responsiveness indicator is stroke volume variation, wherein the instruction to compute a preload responsiveness comprises instructions to:
identify a response pattern in the hemodynamic variable by bandpass filtering the hemodynamic variable;
identify a maximum value (max) from the identified response pattern;
identify a minimum value (min) from the identified response pattern;
calculate a mean value (mean) of the max and the min; and
compute the preload responsiveness indicator as: (max−min)/mean×100.

Example 53. The system of example 52, wherein the max is a largest value of the response pattern.

Example 54. The system of example 52, wherein the max is an average of at least two largest values of the response pattern.

Example 55. The system of example 52, wherein the min is a smallest value of the response pattern.

Example 56. The system of example 52, wherein the min is an average of at least two smallest values of the response pattern.

Example 57. The system of example 34, wherein the instruction to compute a preload responsiveness comprises instructions to:
identify a response pattern in the hemodynamic variable by bandpass filtering the hemodynamic variable, wherein the bandpass filtering possesses a frequency range;
identify a modulation pattern in the waveform, wherein the modulation pattern is associated with a stimulus that affects blood pressure or blood flow;
compute a power density spectrum of the hemodynamic variable from the identified heart beats in the modulation pattern; and
compute the preload responsiveness indicator as a mean value (mean) of an amplitude of the power density spectrum over the frequency range.

Example 58. The system of example 57, wherein the stimulus is selected from one or more of the group consisting of: fluid infusion, vasopressor infusion, vasodilator infusion, spontaneous respiration, and mechanical respiration.

Example 59. The system of example 57, wherein the frequency range is approximately 0.08 Hz to approximately 0.33 Hz.

Example 60. The system of any one of examples 34-59, wherein the plurality of heart beats represents at least one respiratory cycle.

Example 61. The system of any one of examples 34-60, wherein the memory further comprises instructions to provide directions to perform a medical intervention to the patient.

Example 62. The system of any one of examples 34-61, wherein the memory further comprises instructions to perform an automated medical intervention to the patient.

Example 63. The system of example 61 or 62, wherein the medical intervention comprises one or more of administering a fluid bolus, providing mechanical ventilation, or administering a pharmacological agent.

Example 64. The system of example 63, wherein the pharmacological agent is selected from a vasopressor and a vasodilator.

1. A hemodynamic monitoring system for determining a preload responsiveness indicator, comprising:
a display;
a sensory alarm;
a right-heart catheter with a port located within a tip portion of the right-heart catheter, the tip portion configured to be located within a pulmonary artery of a patient;
a pressure or flow sensor configured to sense a right-heart blood pressure or flow of the patient through a lumen in communication with the port; and
a computational system in communication with the pressure or flow sensor, the computational system comprising at least one processor and a memory comprising instructions that when executed instruct the computational system to:
generate a waveform from a patient's right heart circulation in real-time, wherein the waveform comprises a plurality of heart beats, and wherein the waveform is a blood pressure waveform or a blood flow waveform;
identify individual heart beats in the waveform;
obtain a hemodynamic variable for each identified heart beat in the waveform; and
compute a preload responsiveness indicator using the obtained hemodynamic variable.

2. A hemodynamic monitoring system for determining a preload responsiveness indicator, comprising:
a pressure or flow sensor configured to sense a right-heart blood pressure or flow of a patient; and
a computational system in communication with the pressure or flow sensor, the computational system comprising at least one processor and a memory comprising instructions that when executed instruct the computational system to:
generate a waveform from a patient's right heart circulation in real-time, wherein the waveform comprises a plurality of heart beats, and wherein the waveform is a blood pressure waveform or a blood flow waveform;
identify individual heart beats in the waveform;
obtain a hemodynamic variable for each identified heart beat in the waveform; and
compute a preload responsiveness indicator using the obtained hemodynamic variable.

3. The system of claim 2, wherein the instruction to generate the waveform comprises an instruction to:
sense blood pressure or blood flow from at least one of: a right ventricle, a right atrium, a pulmonary artery, and a pulmonary vein; or
derive blood pressure or blood flow representative of: a right ventricle, a right atrium, a pulmonary artery, and a pulmonary vein.

4. The system of claim 2, wherein sensing blood pressure or blood flow comprises the use the pressure or flow sensor.

5. The system of claim 2, wherein the waveform is a blood flow waveform of a right ventricle or a pulmonary artery.

6. The system of claim 5, wherein the pressure or flow sensor is an invasive ultrasound probe to sense blood flow.

7. The system of claim 2, wherein the waveform is a blood flow waveform computed from a right ventricular blood pressure waveform or a pulmonary artery blood pressure waveform.

8. The system of claim 7, wherein the blood flow waveform is computed using a model of an input impedance of pulmonary circulation.

9. The system of claim 8, wherein the model of the input impedance of the pulmonary circulation is based on a physiological Windkessel model.

10. The system of claim 2 wherein the hemodynamic variable is selected from the group consisting of: right ventricular stroke volume, right ventricular pulse pressure, right ventricular systolic pressure, pulmonary artery pulse pressure, and pulmonary artery systolic pressure.

11. The system of claim 10, wherein the obtained hemodynamic variable is right ventricular stroke volume, wherein right ventricular stroke volume is defined as a standard deviation of pulmonary arterial pressure of that beat.

12. The system of claim 11, wherein the hemodynamic variable is right ventricular stroke volume, wherein right ventricular stroke volume for each beat is defined as a standard deviation of pulmonary arterial pressure of that beat normalized by a mean pulmonary arterial pressure of that beat.

13. The system of claim 2, wherein the instruction to obtain a hemodynamic variable comprises an instruction to filter the hemodynamic variables.

14. The system of claim 13, wherein the instruction to filter comprises one or more instruction selected from:
remove beats whose values of the hemodynamic variable are at least two standard deviations greater than or at least two standard deviations below its mean;
remove beats whose values of the hemodynamic variable are in a highest 5% of data; or
remove beats whose values of the hemodynamic variables are in a lowest 5% of data.

15. The system of claim 2, wherein the preload responsiveness indicator is stroke volume variation, wherein the instruction to compute a preload responsiveness comprises instructions to:
identify a maximum value (max) of the hemodynamic variable;
identify a minimum value (min) of the hemodynamic variable;
calculate a mean value (mean) of the max and the min; and
compute the preload responsiveness indicator as:
$$\frac{max - min}{mean} \times 100 .$$

16. The system of claim 15, wherein the max is a largest value of the hemodynamic variable.

17. The system of claim 15, wherein the max is an average of at least two largest values of the hemodynamic variable.

18. The system of claim 15, wherein the min is a smallest value of the hemodynamic variable.

19. The system of claim 15, wherein the min is an average of at least two smallest values of the hemodynamic variable.

20. The system of claim 2, wherein the preload responsiveness indicator is stroke volume variation, wherein the instruction to compute a preload responsiveness comprises instructions to:
identify a response pattern in the hemodynamic variable by bandpass filtering the hemodynamic variable;
identify a maximum value (max) from the identified response pattern;
identify a minimum value (min) from the identified response pattern;
calculate a mean value (mean) of the max and the min; and
compute the preload responsiveness indicator as:
$$\frac{max - min}{mean} \times 100 .$$

21. The system of claim 20, wherein the max is a largest value of the response pattern or the max is an average of at least two largest values of the response pattern.

22. The system of claim 20, wherein the min is a smallest value of the response pattern.

23. The system of claim 20, wherein the min is an average of at least two smallest values of the response pattern.

24. The system of claim 1, wherein the instruction to compute a preload responsiveness comprises instructions to:
identify a response pattern in the hemodynamic variable by bandpass filtering the hemodynamic variable, wherein the bandpass filtering possesses a frequency range;
identify a modulation pattern in the waveform, wherein the modulation pattern is associated with a stimulus that affects blood pressure or blood flow;
compute a power density spectrum of the hemodynamic variable from the identified heart beats in the modulation pattern; and
compute the preload responsiveness indicator as a mean value (mean) of an amplitude of the power density spectrum over the frequency range.

25. The system of claim 24, wherein the stimulus is selected from one or more of the group consisting of: fluid infusion, vasopressor infusion, vasodilator infusion, spontaneous respiration, and mechanical respiration.

26. The system of claim 24, wherein the frequency range is approximately 0.08 Hz to approximately 0.33 Hz.

27. The system of claim 2, wherein the plurality of heart beats represents at least one respiratory cycle.

28. The system of claim 2, wherein the memory further comprises instructions to provide directions to perform a medical intervention to the patient.

29. The system of claim 2, wherein the memory further comprises instructions to perform an automated medical intervention to the patient.

30. The system of claim 28, wherein the medical intervention comprises one or more of administering a fluid bolus, providing mechanical ventilation, or administering a pharmacological agent.

31. The system of claim 30, wherein the pharmacological agent is selected from a vasopressor and a vasodilator.
